(19) United States
(12) Patent Application Publication
(10) Pub. No.: US 2025/0204846 A1
(43) Pub. Date: Jun. 26, 2025

(54) ALZHEIMER'S DISEASE DIAGNOSIS METHOD AND DIAGNOSIS SYSTEM BASED ON NEURAL POPULATION SIGNAL SYNCHRONIZATION

(71) Applicant: Seoul National University R&DB Foundation, Seoul (KR)

(72) Inventors: Jeehyun KWAG, Seoul (KR); Yoonsoo YEO, Seoul (KR)

(73) Assignee: Seoul National University R&DB Foundation, Seoul (KR)

(21) Appl. No.: 18/988,527

(22) Filed: Dec. 19, 2024

(30) Foreign Application Priority Data
Dec. 20, 2023 (KR) 10-2023-0187505
Sep. 30, 2024 (KR) 10-2024-0133154

(51) Int. Cl. A61B 5/00; A61B 5/293; A61B 5/372; G16H 50/20; G16H 50/30

(52) U.S. Cl. CPC A61B 5/4088; A61B 5/0071; A61B 5/293; A61B 5/372; A61B 5/7271; G16H 50/20; G16H 50/30

(57) ABSTRACT

The present disclosure relates to a Alzheimer's disease diagnosis method based on neural population spike signal synchronization, an Alzheimer's disease diagnosis system, and a Alzheimer's disease diagnosis device based on neural population spike signal synchronization including the system, and it has the advantage of being able to be used for the early diagnosis of Alzheimer's disease by calculating an index for evaluating a degree of neural population signal synchronization of neurons and utilizing the same. In addition, the Alzheimer's disease diagnosis method of the present disclosure is based on the fields of neuroscience, medical imaging and brain disease research, and thus provides information related to dementia by analyzing the neural population spike signal synchronization through a more precise method than the existing method, thereby enabling early diagnosis before Alzheimer's disease symptoms appear, and thus, it can be utilized in various medical and research institutions.

### CROSS-REFERENCE TO RELATED APPLICATION(S)

This application claims priority to and the benefit of Korean Patent Application No. 10-2023-0187505, filed on Dec. 20, 2023, and Korean Patent Application No. 10-2024-0133154, filed on Sep. 30, 2024, the disclosures of which are incorporated herein by reference in its entirety.

### TECHNICAL FIELD

The present disclosure relates to a method for diagnosing Alzheimer's disease based on neural population signal synchronization, a system for diagnosing Alzheimer's disease, and a device for diagnosing Alzheimer's disease based on neural population signal synchronization, including the system.

### RELATED ART

Dementia is mainly divided into Alzheimer's disease and vascular dementia. Alzheimer's disease is characterized by a decline in memory and learning functions due to brain cell damage, whereas vascular dementia is characterized by a decline in cognitive ability due to problems with the cerebral blood vessels.

Alzheimer's disease is characterized by neurological abnormalities and cognitive functional impairments centered on the prefrontal lobe. Pathologically, it is caused by a decrease in acetylcholine, which is a neurotransmitter responsible for communication between brain cells, and abnormal accumulation of Aβ plaques, which leads to damage and cell death of nerve cells and atrophy of brain tissue.

The cerebral cortex plays an important role in regulating higher cognitive functions such as memory, judgment and reasoning. In Alzheimer's disease, neuronal damage and inflammation occur in the cerebral cortex, which leads to a decline in memory function and mental changes. In particular, it plays an important role in storing long-term memories, and in Alzheimer's disease, memory decline occurs as the function of the cerebral cortex is damaged.

Cognitively, short-term memory impairment is mainly observed in the early stages, and as it progresses, various brain functions such as abstract thinking, language ability, and spatial cognition are affected. This results in decreased function in daily life and decreased social adaptability. Alzheimer's disease is one of the main causes of dementia, and early diagnosis and management are important.

However, most of the current dementia diagnosis methods focus on measuring structural changes or blood flow in the brain, thereby making it impossible to diagnose before pathological changes occur, and since it is often performed after symptoms appear, early diagnosis is necessary.

Accordingly, as a result of research to develop a method for diagnosing Alzheimer's disease, the inventors of the present disclosure completed the present disclosure by confirming damage to the synchronized neural signals of cerebral cortex neurons in an Alzheimer's disease mouse model.

### SUMMARY

An object of the present disclosure is to provide a method for diagnosing Alzheimer's disease based on neural population signal synchronization, including collecting population neural activity signals of cerebral cortex neurons; calculating a synchronization index from the collected signals; and diagnosing Alzheimer's disease through the calculated index.

Another object of the present disclosure is to provide a system for diagnosing Alzheimer's disease based on neural population signal synchronization, including a collection part configured to collect neural population activity signals of cerebral cortex neurons; a calculation part configured to calculate a synchronization index from the collected signals; and a judgment part configured to diagnose Alzheimer's disease through the calculated index.

Still another object of the present disclosure is to provide a device for diagnosing Alzheimer's disease based on neural population signal synchronization, including the system

The present disclosure relates to a method for diagnosing Alzheimer's disease by analyzing the synchronization of neural population signals of cortical cells in normal mice and Alzheimer's disease mice by utilizing in vivo calcium imaging, and has the advantage of improved accuracy and early diagnosis compared to conventional techniques by quantitatively and precisely analyzing the difference in cell synchronization between normal and Alzheimer's disease mouse models.

One embodiment of the present disclosure confirmed that the synchronized neural population activity signal seen in the control group C57BL/6J mice was damaged in the 5XFAD mice, which are Alzheimer's disease mouse models.

Hereinafter, the present disclosure will be described in more detail.

Specific structural and functional descriptions of the embodiments of the present disclosure are merely illustrative for the purpose of explaining the embodiments according to the present disclosure, and unless otherwise defined, all terms used herein, including technical or scientific terms, have the same meanings as generally understood by a person having ordinary skill in the art to which the present disclosure pertains. Terms defined in commonly used dictionaries should be interpreted as having meanings consistent with the meanings they have in the context of the relevant technology, and are preferably not interpreted in an ideal or overly formal sense unless explicitly defined in the present disclosure.

The present disclosure provides a method for diagnosing Alzheimer's disease based on neural population signal synchronization, including collecting neural population activity signals of cerebral cortex neurons; calculating a synchronization index from the collected signals; and diagnosing Alzheimer's disease through the calculated index.

In the present disclosure, FIG. 3 is a flowchart illustrating the method for diagnosing Alzheimer's disease based on neural population signal synchronization according to a preferred embodiment of the present disclosure.

Although each step described in the present disclosure is described as being performed by a computer, the subject of each step is not limited thereto, and at least some of each step may be performed in different devices depending on the embodiment.

Alzheimer's disease is one of the main causes of dementia, and early diagnosis and management are important. However, most of the current dementia diagnostic methods focus on measuring structural changes or blood flow in the brain, thereby making it impossible to diagnose before pathological changes occur. In addition, since diagnosis is often made after symptoms appear, early diagnosis is necessary. Accordingly, although Alzheimer's disease symptoms differ from person to person depending on the progression, since an increase in synchronized neural population activity of prefrontal cortex neurons is related to the recall of long-term memory, the synchronized neural population activity signals may be used as an index for dementia judgment by considering these factors.

In one embodiment of the present disclosure, by the method for diagnosing Alzheimer's disease based on neural population signal synchronization, it is possible to collecting neural population activity signals of cerebral cortex neurons, calculate a synchronization index from the collected signals, and diagnose Alzheimer's disease through the calculated index such that it may diagnose whether a user has Alzheimer's disease. Preferably, the method for diagnosing Alzheimer's disease according to the present disclosure calculates a synchronization index from the collected signals according to the collection of neural population activity signals of cerebral cortex neurons for the user, images the calculated index to generate an index image, and analyzes the index image using a pre-learned artificial intelligence model, thereby diagnosing whether the user has Alzheimer's disease.

In the present disclosure, the collecting neural population activity signals from the cerebral cortex neurons may include converting a fluorescent signal of each neural cell into a time series signal from an image measured through a fluorescence microscope.

In the present disclosure, the fluorescent signal of each neural cell may be generated through genetically encoded calcium indicators expressed in neurons.

In the present disclosure, the calculating a synchronization index may include calculating a similarity (SR) index indicating a degree of synchronization of the collected neural population activity signals.

In the present disclosure, the calculation of the similarity index may use Mathematical Formula 1 below.

$$SR = \frac{c ( y \vert x ) + c ( x \vert y )}{{M}_{x} {M}_{y}} , c ( x \vert y ) = {\sum }_{i = 1}^{{M}_{x}} {\sum }_{j = 1}^{{M}_{y}} {J}_{ij} , {J}_{ij} = \{ \begin{matrix}1 if < {s}_{i}^{x} - {s}_{j}^{y} < τ \\ \frac{1}{2} if {s}_{i}^{x} = {s}_{j}^{y} \\ 0 else\end{matrix} \qquad [ Mathematical Formula 1 ]$$

In Mathematical Formula 1, Sᵢ^{x}, Sⱼ^{y} are event occurrence times of an i^{th} or j^{th} neural activity signal of neuron x or y, τ is a maximum time difference of synchronized events, which is half of a minimum time that two neural activity signal events can occur, Mₓ, M_{y} are event counts of neural activity signals of neuron x or y, C(x|y) is a number of times a corresponding event occurs in neuron x within a given time range τ after an event occurs in neuron y, and C(y|x) is a number of times a corresponding event occurs in neuron y within τ after an event occurs in neuron x.

Meanwhile, the result data according to the diagnosis of Alzheimer's disease may be connected to an external device, which provides various information/data necessary for performing a method for diagnosing Alzheimer's disease using the correlation between the collection of neural population activity signals and the calculation of an synchronization index from the collected signals, or can receive, store and manage the result data derived by performing a method for diagnosing Alzheimer's disease using the correlation between the collection of neural population activity signals and the calculation of a synchronization index from the collected signals.

In the present disclosure, the external device may be separately provided outside the device for performing the method for diagnosing Alzheimer's disease, but is not limited thereto.

Hereinafter, the hardware configuration of the device for performing the Alzheimer's disease diagnosis method using the correlation between the collection of neural population activity signals and the calculation of a synchronization index from the collected signals will be described.

The methods or each step described in relation to the embodiments of the present disclosure may be implemented directly as hardware, implemented as a software module executed by hardware, or implemented by a combination of the same. The software module may be stored in a RAM (Random Access Memory), a ROM (Read Only Memory), an EPROM (Erasable Programmable ROM), an EEPROM (Electrically Erasable Programmable ROM), a flash memory, a hard disk, a removable disk, a CD-ROM or any form of a computer-readable recording medium well known in the technical field to which the present disclosure pertains.

The components of the present disclosure may be implemented as a program (or application) to be executed by being combined with a computer, which is hardware, and stored in a medium. The components of the present disclosure may be implemented as software programming or software elements, and similarly, the embodiments may be implemented in programming or scripting languages such as C, C++, Java, assembler and the like, including various algorithms implemented as a combination of data structures, processes, routines, or other programming components.

In addition, the present disclosure provides a system for diagnosing Alzheimer's disease based on neural population signal synchronization, including a collection part configured to collect neural population activity signals of cerebral cortex neurons; a calculation part configured to calculate a synchronization index from the collected signals; and a judgment part configured to diagnose Alzheimer's disease through the calculated index.

Since the corresponding technical features included in the system for diagnosing Alzheimer's disease can be replaced in the above-described part, the description thereof will be omitted.

In the present disclosure, the collection part configured to collect neural population activity signals of cerebral cortex neurons may include a conversion part configured to convert a fluorescence signal of each neuron into a time series signal from an image measured through a fluorescence microscope. According to one embodiment of the present disclosure, the fluorescent signal of each neuron may be generated through a genetically encoded calcium indicator expressed in the neuron.

In the present disclosure, the calculation part configured to calculate a synchronization index may include a processing part that is configured to calculate a similarity (SR) index indicating a degree of synchronization of the collected neural population activity signals. The calculation part according to one embodiment of the present disclosure may calculate the similarity index using Mathematical Formula 1 below.

$$SR = \frac{c ( y \vert x ) + c ( x \vert y )}{{M}_{x} {M}_{y}} , c ( x \vert y ) = {\sum }_{i = 1}^{{M}_{x}} {\sum }_{j = 1}^{{M}_{y}} {J}_{ij} , {J}_{ij} = \{ \begin{matrix}1 if < {s}_{i}^{x} - {s}_{j}^{y} < τ \\ \frac{1}{2} if {s}_{i}^{x} = {s}_{j}^{y} \\ 0 else\end{matrix} \qquad [ Mathematical Formula 1 ]$$

In Mathematical Formula 1, Sᵢ^{x}, Sⱼ^{y} are event occurrence times of an i^{th} or j^{th} neural activity signal of neuron x or y, τ is a maximum time difference of synchronized events, which is half of a minimum time that two neural activity signal events can occur, Mₓ, M_{y} are event counts of neural activity signals of neuron x or y, C(x|y) is a number of times a corresponding event occurs in neuron x within a given time range τ after an event occurs in neuron y, C(y|x) is a number of times a corresponding event occurs in neuron y within τ after an event occurs in neuron x, M^{x,y} is a number of events of the neural activity signal of neuron x or y, and S_{i,j}^{x,y} is an occurrence time of an i^{th} or j^{th} neural activity signal event of neuron x or y.

In addition, the present disclosure provides a device for diagnosing Alzheimer's disease based on neural population signal synchronization, including the system.

The present disclosure relates to a method for diagnosing Alzheimer's disease based on neural population signal synchronization, and has the advantage of being able to calculate an index for evaluating the degree of neural population signal synchronization of neurons and use the same to make an early diagnosis of Alzheimer's disease. In addition, the Alzheimer's disease diagnosis method of the present disclosure is based on the fields of neuroscience, medical imaging and brain disease research, and thus provides information related to dementia by analyzing the neural population signal synchronization through a more precise method than the existing method, thereby enabling early diagnosis before Alzheimer's disease symptoms appear, and thus, it can be utilized in various medical and research institutions.

### BRIEF DESCRIPTION OF THE DRAWINGS

FIG. 1 shows the process of measuring a degree of synchronization of neural population activity of prefrontal cortex neurons in normal mice and Alzheimer's disease mouse models.

FIG. 2 shows the results of measuring a degree of synchronization of neural population activity of prefrontal cortex neurons in normal mice and Alzheimer's disease mouse models.

FIG. 3 is a flowchart illustrating a method for diagnosing Alzheimer's disease based on neural population signal synchronization according to the present disclosure step by step.

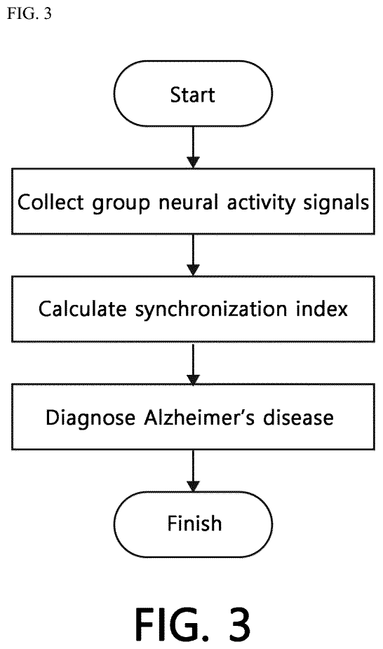

### DETAILED DESCRIPTION OF THE EMBODIMENTS

Hereinafter, in order to help understand the present disclosure, examples will be provided to explain in detail. However, the following examples are only intended to illustrate the content of the present disclosure, and the scope of the present disclosure is not limited to the following examples. The examples of the present disclosure are provided to more completely explain the present disclosure to those with ordinary skill in the art.

### Example 1. Experimental Animal Model

The present disclosure used 5XFAD mice as an Alzheimer's disease mouse model, and these were 6-7-month-old mice obtained from Jackson Laboratory, USA (#34840-JAX). C57BL/6J mice were used as a normal mouse control group of the 5XFAD mouse group. Only male 5XFAD and C57BL/6J were used in the behavioral experiment, and the genotype of the 5XFAD mouse was confirmed through polymerase chain reaction (PCR) using genomic DNA extracted from tail samples. All mice were housed individually in temperature- and humidity-controlled vivarium and maintained on a 12-hour backlight cycle (lights on at 5 p.m. and off at 5 a.m.). All experiments were performed during the dark period, and food and water were provided ad libitum.

### Example 2. In Vivo Calcium Imaging

Normal and Alzheimer's disease mice were anesthetized with 2% isoflurane (2 to 2.5 mL/min) and fixed for stereotaxic surgery. Four screws were inserted around the implantation site, and a hole was made in the skull using a dental drill. Afterwards, 500 nL of AAV-CaMKII-GCaMP6s virus was injected into the right prefrontal cortex (PFC) using a Hamilton syringe (Hamilton Company, Reno, NV). After virus injection, a 1-mm diameter and 4.0-mm long Gradient Index (GRIN) lens (Inscopix, Palo Alto, CA) was implanted over the injection site and secured to the skull using dental cement. After a 2-week recovery period, mice were examined for GCaMP expression using a miniature fluorescence microscope (nVoke, Inscopix, Palo Alto, CA), after which a baseplate was cemented over the implanted GRIN lens to maintain a consistent focal plane for all subsequent imaging sessions, and these were used for measurements of the neural population activity of prefrontal cortex neurons.

### Example 3. Contextual Fear Conditioning Protocol

In a behavioral task, mice undergo a contextual fear memory paradigm for 15 days, concurrent with measurements of prefrontal cortex population activity using calcium imaging. During habituation, mice were exposed to a standard fear conditioning chamber (Coulbourn Instruments, Holliston, MA) with specific contextual cues: 30 cm (length)×25 cm (width)×33 cm (height). Afterwards, during subsequent conditioning, mice were exposed to the same context for 8 minutes, and 2 minutes after being placed in the chamber, mice received three 1-second shocks at 0.5 mA intensity that were spaced 2 minutes apart. During the fear memory recall phase, mice were re-exposed to the same context for 3 minutes while recalling the contextual fear memory, and freezing behavior was assessed. After each session, the fear chamber was cleaned with 70% ethanol, and the behavior of the mice was recorded using Etho Vision XT 16 video software (Noldus, Leesburg, VA), and freezing behavior was quantified based on pixel changes in the recorded images using the same software.

### Example 4. Confirmation of Damage to Synchronized Population Neural Activity Signals in Alzheimer's Disease Mouse Model

In order to investigate the difference in synchronized population neural activity signals in Alzheimer's disease mouse model, a protocol capable of measuring population neural activity of the prefrontal cortex during contextual fear conditioning protocol was established.

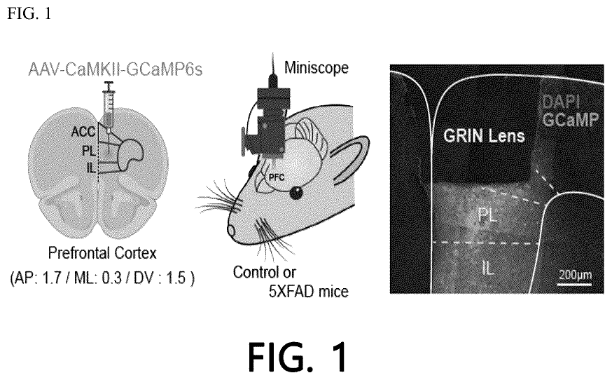

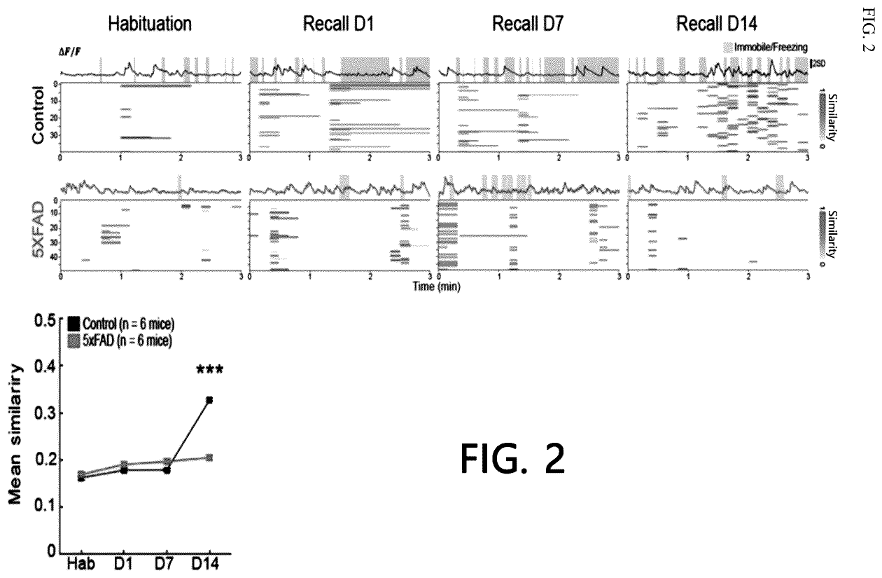

The degree of synchronization of neural population activity of prefrontal cortex neurons was measured in normal mice and Alzheimer's disease mouse model, and the results are shown in FIGS. 1 and 2. As shown in FIGS. 1 and 2, it was confirmed that an increase in synchronized neural population activity in long-term memory recall seen in normal mice was impaired in the Alzheimer's disease mouse model.

The above results mean that an increase in synchronized neural population activity of prefrontal cortex neurons is related to long-term memory recall, and Alzheimer's disease can be diagnosed through the index of synchronized group neurons of the present disclosure.

While the specific parts of the present disclosure have been described in detail above, it is apparent to those skilled in the art that such specific description is merely a preferred embodiment and the scope of the present disclosure is not limited thereby. In other words, the actual scope of the present disclosure is defined by the appended claims and their equivalents.

What is claimed is:

1. A method for diagnosing Alzheimer's disease based on neural population signal synchronization, the method comprising:
collecting neural population activity signals of cerebral cortex neurons;
calculating a synchronization index from the collected signals; and
diagnosing Alzheimer's disease through the calculated index.

2. The method of claim 1, wherein the collecting neural population activity signals from cerebral cortex neurons comprises converting a fluorescent signal of each neuron into a time series signal from an image measured through a fluorescence microscope, or acquiring using a neural signal measurement technique through an implanted electrode.

3. The method of claim 2, wherein the fluorescent signal of each neuron is generated through genetically encoded calcium indicators expressed in neurons, or acquiring using a neural signal measurement technique through an implanted electrode.

4. The method of claim 1, wherein the calculating a synchronization index comprises calculating a similarity (SR) index indicating a degree of synchronization of the collected neural population activity signals.

5. The method of claim 4, wherein the calculating a similarity index uses Mathematical Formula 1 below:
$$SR = \frac{c ( y \vert x ) + c ( x \vert y )}{{M}_{x} {M}_{y}} , c ( x \vert y ) = {\sum }_{i = 1}^{{M}_{x}} {\sum }_{j = 1}^{{M}_{y}} {J}_{ij} , {J}_{ij} = \{ \begin{matrix}1 if < {s}_{i}^{x} - {s}_{j}^{y} < τ \\ \frac{1}{2} if {s}_{i}^{x} = {s}_{j}^{y} \\ 0 else\end{matrix} \qquad [ Mathematical Formula 1 ]$$
wherein in Mathematical Formula 1, Sᵢ^{x}, Sⱼ^{y} are event occurrence times of an i^{th} or j^{th} neural activity signal of neuron x or y,
τ is a maximum time difference of synchronized events, which is half of a minimum time that two neural activity signal events can occur,
Mₓ, M_{y} are event counts of neural activity signals of neuron x or y,
C(x|y) is a number of times a corresponding event occurs in neuron x within a given time range τ after an event occurs in neuron y,
C(y|x) is a number of times a corresponding event occurs in neuron y within τ after an event occurs in neuron x,
M^{x,y} is a number of events of the neural activity signal of neuron x or y, and
S_{i,j}^{x,y} is an occurrence time of an i^{th} or j^{th} neural activity signal event of neuron x or y.

6. A system for diagnosing Alzheimer's disease based on neural population signal synchronization, comprising:
a collection part configured to collect neural population activity signals of cerebral cortex neurons;
a calculation part configured to calculate a synchronization index from the collected signals; and
a judgment part configured to diagnose Alzheimer's disease through the calculated index.

7. The system of claim 6, wherein the collection part configured to collect neural population activity signals of cerebral cortex neurons comprises a conversion part that is configured to convert a fluorescence signal of each neuron into a time series signal from an image measured through a fluorescence microscope.

8. The system of claim 7, wherein the fluorescent signal of each neuron is generated through genetically encoded calcium indicators expressed in neurons.

9. The system of claim 6, wherein the calculation part configured to calculate a synchronization index comprises a processing part that is configured to calculate a similarity (SR) index that indicates a degree of synchronization of the collected neural population activity signals.

10. The system of claim 9, wherein the calculating a similarity index uses Mathematical Formula 1 below:
$$SR = \frac{c ( y \vert x ) + c ( x \vert y )}{{M}_{x} {M}_{y}} , c ( x \vert y ) = {\sum }_{i = 1}^{{M}_{x}} {\sum }_{j = 1}^{{M}_{y}} {J}_{ij} , {J}_{ij} = \{ \begin{matrix}1 if < {s}_{i}^{x} - {s}_{j}^{y} < τ \\ \frac{1}{2} if {s}_{i}^{x} = {s}_{j}^{y} \\ 0 else\end{matrix} \qquad [ Mathematical Formula 1 ]$$
wherein in Mathematical Formula 1, Sᵢ^{x}, Sⱼ^{y} are event occurrence times of an i^{th} or j^{th} neural activity signal of neuron x or y,
τ is a maximum time difference of synchronized events, which is half of a minimum time that two neural activity signal events can occur,
Mₓ, M_{y} are event counts of neural activity signals of neuron x or y,
C(x|y) is a number of times a corresponding event occurs in neuron x within a given time range τ after an event occurs in neuron y,
C(y|x) is a number of times a corresponding event occurs in neuron y within τ after an event occurs in neuron x,
M^{x,y} is a number of events of the neural activity signal of neuron x or y, and
S_{i,j}^{x,y} is an occurrence time of an i^{th} or j^{th} neural activity signal event of neuron x or y.

11. A device for diagnosing Alzheimer's disease based on neural population signal synchronization, comprising the system according to claim 6.
